(19) United States
(12) Patent Application Publication
(10) Pub. No.: US 2025/0090322 A1
(43) Pub. Date: Mar. 20, 2025

(54) AN EXTERNAL AORTIC ANNULOPLASTY RING AND A METHOD OF MANUFACTURING SAME

(71) Applicants: Aarhus Universitet, Aarhus C (DK); Region Midtjylland, Viborg (DK)

(72) Inventors: Mariam Abdi Noor, Aarhus C (DK); John Michael Hasenkam, Aarhus C (DK); Leila Louise Benhassen, Aarhus C (DK); Peter Johansen, Aarhus C (DK)

(21) Appl. No.: 18/291,787

(22) PCT Filed: Jul. 29, 2022
(86) PCT No.: PCT/EP2022/071334

§ 371 Date: Jan. 24, 2024

(30) Foreign Application Priority Data
Jul. 30, 2021 (EP) 21188701.3

(51) Int. Cl. A61F 2/24

(52) U.S. Cl. CPC A61F 2/2445; A61F 2210/0009; A61F 2210/0057; A61F 2220/0008; A61F 2220/0025; A61F 2240/002; A61F 2250/0018; A61F 2250/0037

(57) ABSTRACT

The present invention concerns an external aortic annuloplasty ring for positioning around the circumference of an aortic valve for external aortic root repair or stabilization of the annulus to support the aortic valve, wherein the ring is open-ended and thereby having two opposite open ends, wherein the ring comprises at least two sections with different elastic properties along the perimeter of the ring, and wherein said opposite open ends are suitable for being joined together, such as by suturing, so as to form a closed ring around the aortic root. The invention further concerns a method for manufacturing such a ring.

### FIELD OF THE INVENTION

Aspects of the present invention relate to an external aortic annuloplasty ring and methods of manufacturing and using the same.

### BACKGROUND OF THE INVENTION

In recent years, aortic valve repair is increasingly gaining more ground as an alternative to aortic valve replacement. This is to avoid complications related to prosthetic replacement such as the increased risk of infection, lifelong medication, and high annual risk of haemorrhage.

When performing aortic valve repair, it has been acknowledged that stabilizing while preserving the dynamics of the aortic valve plays a pivotal role for the repair and is a contributing factor for delivering better outcomes. Therefore, it is essential to investigate how to properly preserve and strengthen the aortic annulus while maintaining the natural dynamics of the native aortic root. The aortic annulus dynamically expands during the cardiac cycle and studies have shown that there are segmental differences of this expansion in the native aortic root and during valve-sparring techniques with and without supportive annuloplasty. This is linked to the heterogeneous composition and anatomical structure of the aortic annulus. Hence, various challenges remain as the preservation and repair of aortic valves evolves as a discipline including the advancement of materials and techniques for a reliable aortic valve annuloplasty.

An external annuloplasty ring at the aortic annulus together with remodelling procedures are now part of the guidelines for the management of valvular heart disease, by the European Society of Cardiology and European Association of Cardio-Thoracic Surgery, in young patients with aortic root dilation.

The design of an external annuloplasty ring has over the years been subject to a number of studies. For instance a study of an external annuloplasty ring implanted in sheep is disclosed in the article entitled “An expansible aortic ring for a physiological approach to conservative aortic valve surgery” (Lansac E, Di Centa I, Raoux F, Bulman-Fleming N, Ranga A, Abed A, Ba M, Paolitto A, Letourneur D, Meddahi-Pellé A. An expansible aortic ring for a physiological approach to conservative aortic valve surgery. J Thorac Cardiovasc Surg. 2009 September; 138 (3): 718-24. doi: 10.1016/j.jtcvs.2009.05.024. PMID: 19698861). The ring is a Coroneo annuloplasty ring, as disclosed in US2013/0073033A1. The researchers modified the ring, by cutting through the cross-section of the ring for easier implantation unto the aortic root, by passing an open end of the ring under the coronary arteries. After positioning of the ring, the ring was sewn onto the annular base level at five points.

U.S. Pat. No. 10,561,499 B2 discloses an aortic ring and ancillary device for implanting it. In one embodiment disclosed therein, the ring comprises an elastic band, such as a silicone band and an outer fabric layer, wherein said band and fabric is open ended and intended for suturing around the aortic root of a patient, after positioning, so as to form a closed ring around the aortic root.

US 2013/0073033 A1 discloses an expandable annuloplasty ring and associated ring holder. The ring comprises a first and second, spaced apart, elastic core members, and an outer fabric layer, which surrounds each of the first and second core members separately while holding them together around the length of said core members. The core members and the fabric have two free ends, so as to enable a surgeon to position the device around an outer circumference of an aorta, without the need of resecting said aorta.

EP 3 150 173 A1 discloses a reabsorbable subaortic ring. The device comprises an elastic, open-ended ring suitable for positioning within the aorta, by the aortic valve and is closed onto the ring shape, by tying the two loose ends of said ring together. Furthermore, the device is made from a biodegradable material, such as a polydioxanone polymer, so as to be dissolved within a year from implant.

In some patent publications, such as US2021/014558A1 and WO2016/201084A1 there are described annuloplasty bands for the mitral valve. However, the mitral valve does not have a similar anatomical structure as the aortic valve.

Some concerns have been raised regarding the expansibility of the known aortic annuloplasty ring throughout the cardiac cycle, because the material of the tube graft is less compliant than the native tissue and it is also thought to have limited radial expansion capacity. Furthermore, studies have shown the material to expand up to 20% months after implantation.

Accordingly, the need for improved external aortic annuloplasty rings that support the aortic valve by positioning around the circumference of the aortic valve and promote root repair and/or stabilization of the annulus is manifest.

### SUMMARY OF THE INVENTION

Described herein are several embodiments of open-ended annuloplasty rings, which are configured for positioning around the circumference of a blood vessel. In some embodiments, said open-ended annuloplasty rings are configured for external aortic root repair or stabilization of the annulus so as to support the aortic valve, such as for aortic valve repair or replacement. Preferably, the aforementioned open-ended annuloplasty rings comprise an elastic core member, and an outer sheath layer, wherein the elastic core member comprises at least two sections comprising different elastic properties along the perimeter of the ring, preferably by providing non-uniform cross-sections.

Accordingly, some embodiments comprise and open expansible aortic annuloplasty ring and methods of making and using such devices. The open expansible aortic annuloplasty rings described herein are configured to support and stabilize the aortic valve while addressing normal physiological aortic root dynamics. The open expansible aortic annuloplasty rings described herein can be implanted externally around the aortic annulus, which desirably involves less surgery than similar conventional devices.

As set forth herein, it has been realized that the asymmetric dynamics around the aortic annulus and the annular segmental expansion from mid-diastole to mid-systole (MS-MD) is not uniform but rather different around the aorta root. Accordingly, designing the open expansible aortic annuloplasty rings as set forth herein surprisingly was found to provide more benefits than conventional designs including improved support and stabilisation of the aortic valve. In some embodiments, for example, the aortic rings described herein have predetermined differences in the elastic properties along the perimeter of the ring, such as differing sections of elasticity and preferably, these differing sections of elasticity correspond, match, or mirror the asymmetric dynamics, which exist at and around the aortic root.

The aortic annuloplasty rings described herein are preferably stable and possess consistent dynamic annuloplasty that adequately or efficiently downsize the aortic annulus to a desired circumference, and/or increase coaptations length and decrease tenting area and/or geometric orifice area. Furthermore, embodiments described herein have demonstrated acceptable expansibility in vitro as well as in vivo, which is comparable to native aortic root dynamics. Additionally, the heterogeneous design feature of the annuloplasty rings described herein has revealed the capability of a targeted support to specific segments of the aortic annulus.

The external aortic annuloplasty rings provided in this disclosure preferably have similar expansible dynamics as the native aortic valve and desirably mimic the changes during the cardiac cycle by increasing the ring thickness in the areas and aortic valve segments that need more support. The asymmetric design of some of the embodiments of the open expansible aortic annuloplasty rings described herein contribute to this expansible dynamics.

The term “elastic properties” as used in this disclosure is meant a material's ability to stretch or deform when subjected to tensile forces and return to its original shape. In some embodiments, it is beneficial that the core member of the open expansible aortic annuloplasty ring can be subjected to at least a 20% or at least about a 20% strain and return to its original length e.g., at least a 10%, 11%, 12%, 13%, 14%, 15%, 16%, 17%, 18%, 19%, 20%, 21%, 22%, 23%, 24%, 25%, 26%, 27%, 28%, 29%, or 30% strain or a strain that is within a range defined by any two of the aforementioned values.

To obtain expansibility without compromising the material properties, the ring is preferably produced in silicone and covered with a polyester fabric or an outer layer of another suitable type medical grade polymer.

In some embodiments, the core member and the external sheath are formed as a band comprising two, opposite open ends, suitable for joining together, such as by suturing, so as to form a closed ring around the aortic root. Preferably, the core member has a first length and the sheath has a second length, where the second length of the external sheath is longer than the first length of the elastic core member.

The open-ended ring is preferably preformed within a predetermined diameter.

It is preferred that the core member comprises silicone or another elastic material, which exhibits a low stress creep and also that the outer layer comprises a medical grade polyester, such as polyethylene terephthalate (PET). Many embodiments of the open expansible aortic annuloplasty rings described herein are configured to reduce or inhibit creep, i.e. the tendency of the material to slowly deform permanently under the influence of persistent mechanical stresses.

In order to reduce the radial extension of the ring when positioned around the aorta, for example, the radial thickness of the core member is less that its axial extension, i.e. the height (h).

In some embodiments, the non-uniform cross-sections of the open expansible aortic annuloplasty ring comprises at least two, and optionally more, such as four or six, sections having different elastic properties along the perimeter of the ring. The non-uniform sections can be introduced, for example, by altering the radial thickness (t₁, t₂) of the elastic core member.

In more embodiments, the core member is configured to accommodate the specific aortic valve of a patient, based on a model thereof obtained by a scanning procedure, such as CT scanning, echocardiography or suitable types of image obtaining methods. Thus some of the contemplated methods are practiced by obtaining a computerized image of the aorta e.g., CT scanning, echocardiography or suitable types of image obtaining methods, and configuring the architecture of the open expansible aortic annuloplasty ring and/or its core member to match, mirror, or accommodate the structure or dynamics of a patient's aorta as revealed through said scanning techniques. Accordingly, in some embodiments, the data generated by CT scanning, echocardiography or suitable types of image obtaining methods of a subject's aorta is used to configure the structure and/or design of the open expansible aortic annuloplasty ring and/or its core member and this data can be integrated or imported into modelling software to further refine or design the open expansible aortic annuloplasty ring and/or its core member to accommodate a patient's needs and/or such data can be integrated or imported into manufacturing devices and/or processes such as a 3D printer, which can be used to generate customized aortic annuloplasty rings and/or core members for these rings.

In a second aspect of the invention, there is provided a method for manufacturing an annuloplasty ring for positioning around the circumference of a blood vessel, in particular an open-ended annuloplasty ring for external aortic root repair or stabilization of the annulus, to support the aortic valve, such as for aortic valve repair or replacement, said method comprising:
providing a model of a blood vessel of a patient,
determining the cross-sectional size, in particular the annulus diameter, of said blood vessel based on said model,
manufacturing at least a first elastic core member with a length corresponding to a length of a circumference of said blood vessel, and wherein a cross-sectional diameter of the elastic core member is non-uniform along the length of said elastic core member.

A preferred manufacturing method for an aortic annuloplasty ring is provided herein, whereby the aortic ring is manufactured in accordance to patient specific dimensions of the patient's aortic structure or dynamics, and thereby improving the performance of the aortic ring.

Preferably, the at least two sections having different elastic properties are formed in the elastic core member in accordance to the model of the blood vessel.

In a preferred embodiment, the manufacturing of the elastic core member is accomplished by an added manufacturing process, such as 3D printing. Furthermore, in some embodiments, it is preferred that subsequent to the manufacturing of the elastic core member, an outer sheath layer is provided around the core member, and whereby said outer sheath layer preferably is provided by an additive manufacturing process, such as 3D printing.

### BRIEF DESCRIPTION OF THE FIGURES

In the following the invention is described in more detail with reference to the embodiments shown in the accompanying drawings, in which:

FIG. 1 is a schematic perspective view of an aortic annuloplasty ring according to an embodiment of the invention;

FIG. 2 is a schematic perspective view of an embodiment of an aortic annuloplasty ring according to the invention;

FIG. 3 is a schematic view of an aortic annuloplasty ring according to the invention implanted around the aortic root;

FIG. 4 is a schematic cross-sectional illustration of the heart valve anatomy;

FIG. 5 is a graphic illustration of the annular segmental expansion of the aortic annulus from mid-diastole to mid-systole (MS-MD);

FIG. 6 is a flow diagram of the process of manufacturing an aortic annuloplasty ring according to the invention; and

FIG. 7 shows a desired embodiment of the aortic annuloplasty ring.

FIG. 8 shows a desired embodiment of the aortic annuloplasty ring.

FIG. 9 shows a desired embodiment of the aortic annuloplasty ring.

FIG. 10 shows a desired embodiment of the aortic annuloplasty ring.

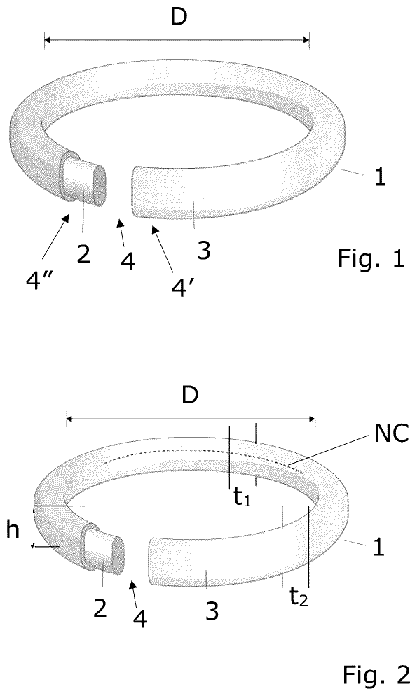

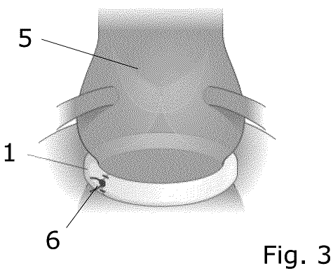

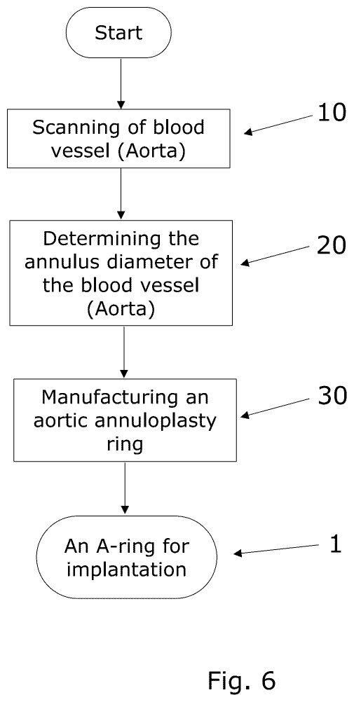

With reference to FIGS. 1 and 2, there is provided an aortic annuloplasty ring 1 with a silicon interior elastic core 2 and polyamide exterior sheath 3. In FIG. 3 there is an illustration of a method for deploying the annuloplasty ring 1 e.g., implantation or deployment externally around the aortic annulus 5. The ring 1 is preferably premade, e.g. by moulding, additive manufacturing (3D printing), or otherwise manufactured, with a pre-shaped annular form with a gap 4 in the ring 1. An example of the manufacturing process is shown in FIG. 6. The core member 1 and the external sheath 2 are formed as an annular band comprising two, opposite open ends 4′, 4″, suitable for joining together, such as by suturing 6, so as to form a closed ring around the aortic root 5 (see FIG. 3). Preferably, the core member 1 has a first length and the sheath 2 has a second length, where the second length of the external sheath 2 is longer than the first length of the elastic core member 1 so that the sheath 2 can completely cover the core member 1 when joined together.

The aortic annuloplasty ring (A-ring) 1 comprised of a silicone core 2 (Essil 291, Axson-sika, Switzerland) covered with a polymer fabric (polyamide 6,6). Silicone is an already well-known implant material and is accordingly found to be the appropriate material due to the desired mechanical and chemical properties it possesses. This includes flexibility, thermal stability, and outstanding biocompatibility. For the outer layer or sheath 3, polyamide is a preferred material as it is a verified implantable textile due to its high tensile strength and general acceptable mechanical properties.

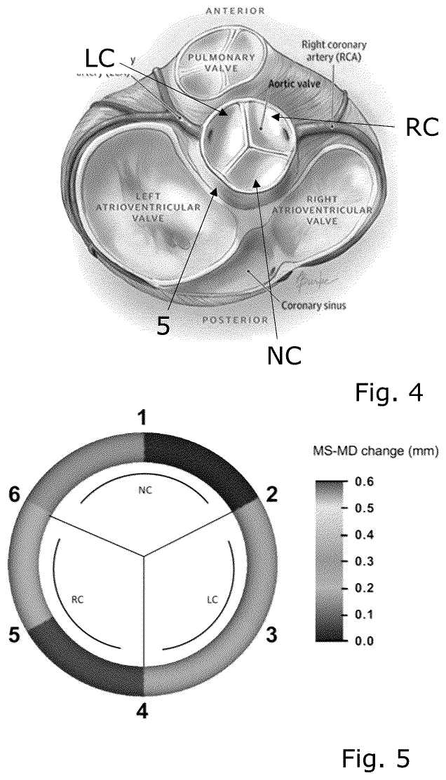

The dimensions of the A-ring 1 are shown in FIG. 2 with an internal diameter D, a height h, and a first and second thickness t₁ and t₂, respectively. The part of the A-ring 1 situated on the non-coronary sinus NC (see FIGS. 4 and 5) at the level of the annulus of the aorta valve had a first thickness t₁, e.g. of 2.6 mm. Based on a geometrical studies, the non-coronary segment NC of the ring 1 was slightly reduced with a smaller thickness compared with the right and left coronary segments RC and LC in order to comply with the asymmetric characteristics of the native aortic annulus 5.

As shown in FIGS. 1 and 2, the ring is open-ended. This allows for the annuloplasty ring 1 to be implanted or deployed externally around the aortic annulus 5, as shown in FIG. 3. The A-ring 1 is sutured together to form a closed ring around the aortic annulus 5. The ring 1 may also be sutured to the tissue of the aortic root of the aortic annulus 5 so that it is ensured that the A-ring 1 is not repositioned after implantation or deployment, which may have an adverse effect as the different elastic properties of the sectors of the A-ring 1 mimic the asymmetric characteristics of the aortic annulus and therefore any angular displacement may jeopardize the intended effect of the A-ring.

The ring 1 is preferably premade, e.g. by moulding additive manufacturing, such as by 3D printing, or otherwise manufactured, with the annular pre-shaped form with a diameter, which is suitable for or configured for the patient in accordance with the patient's aortic architecture and/or dynamics. It is normal to produce a number of implants in different sizes so that the surgeon can choose the most appropriate size of the implant. However, by some approaches set forth herein, initially scanning the patient's aorta using imaging techniques e.g., provides an exact measure or size and topography of the aortic root, which allows a patient custom implant be manufactured and the A-ring 1 may be premade having the appropriate inner diameter corresponding to the diameter of the aortic root of the specific patient.

As shown in FIG. 6, the aorta may be scanned (10) and based on the result of the scanning, the dimensions of the blood vessel, in particular the aorta, may be determined (20), such as the diameter D of the aortic root and the wall thickness. These dimensions are then used to manufacture (30) the annuloplasty ring 1 e.g., by importing the dimensions and aortic architecture parameters into a 3D printer so it is dimensioned in precise correspondence with the specific patient's dimensions.

In FIG. 4 there is shown a cross-sectional illustration of the heart valve anatomy, wherein it can be seen that the aorta valve comprises a non-coronary segment NC, a right coronary segment RC, which is connected to the right coronary artery (RCA), and a left coronary segment RC, which is connected to the left coronary artery (LCA).

In FIG. 5 the result of measurements of the annular segmental expansion from Mid-diastole to Mid-systole (MS-MD) is illustrated in grated colours. It shows a large expansion in the RC and almost no expansion in the NC. It is this asymmetric dynamics around the aortic annulus and the associated heterogeneous forces and geometry that the present invention is addressing.

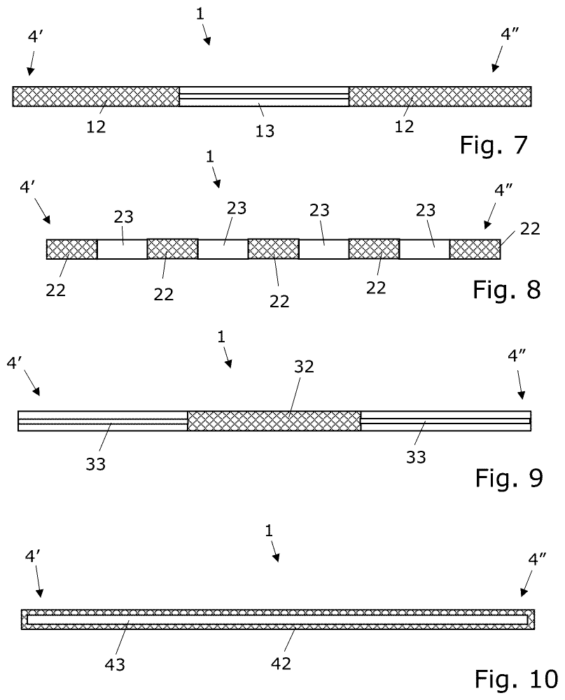

In FIGS. 7 to 10, there are shown three embodiments of the external aortic annuloplasty ring 1 in a straight configuration. The ring 1 has the two opposite open ends 4′ and 4″. In the embodiment of FIG. 7, the ring 1 is made up by three sections where two sections are made of fabric 12 and in between these two fabric sections 12 there is an elastomer section 13. The elastomer section 13 has a different elastic properties, i.e. difference in the elastic modulus, than the fabric sections 12. The fabric sections 12 may be produced with the same elasticity or with different elastic properties.

In FIG. 8, there is shown another embodiment wherein a plurality of fabric sections 22 with elastomer sections 23 between each of the fabric sections 22 is provided. Each of the elastomer sections 23 may be provided with either the same or with different elastic properties. Similarly, the fabric sections 22 may be provided with either the same or with different elastic properties. This allows for providing an asymmetric elasticity along the ring to mimic the asymmetric expansion of the aortic valve during the cardiac cycle.

A further embodiment is shown in FIG. 9, wherein two elastomer sections 33 are provided with a fabric section 32 there between.

In FIG. 10, an example of a ring made of an elastomer section 43 as a core with a fabric section 42 around the core section is provided.

It is realised that other equivalent designs and layouts of the combination of sections may be provided.

The elastomer sections 13, 23, 33, 43 referred to above in the examples of FIGS. 7 to 9, may be made of a silicone elastomer or other elastic material, such as polyvinyl-alcohol based hydrogels or cryogels, polyurethanes, rubber or the like materials suitable for implantation.

The fabric sections 12, 22, 32, 42 may be made of a fabric suitable for implantation and with a weaving pattern that provides some flexibility so that sections can expand and retract in accordance with the cardiac cycle.

The flexibility and the elasticity of the elastomer sections 13, 23, 33 as well as the fabric sections 12, 22, 32 may be provided by various design configurations, which may include varying material thickness, material density, and/or cross-sectional shapes.

At the ring ends 4′ and 4″ the sections are preferably made of a material, which is suitable for being joined together by suturing or the like.

### EXAMPLE

In an example, ring 1 having the dimensions: internal diameter D of 22 mm, height of 4 mm, a first thickness t₁ of 2.6 mm and a second thickness t₂ of 2.8 mm. The size of the A-ring 1 was designed to fit the aortic annulus of an 80 kg porcine subject.

In the example, the ring 1 was made similar to the embodiment of FIG. 1 in a mould and the silicone core was produced in poly (acrylonitrile/butadiene/styrene) (ABS, RenShape® SL 7810, 3D Systems, USA) using selective laser sintering (Projet 7000 HD, 3D Systems, USA). The liquid silicone was poured into the mould with a syringe and stored at room temperature for 48 hours. Subsequently the solid silicone ring was covered in a polyamide fabric and forming the complete A-ring.

The invention is described above with reference to some currently preferred embodiments. However, by the invention it is realised that other embodiments and variants may be provided without departing from the scope of the invention as defined in the accompanying claims. As an example it is realised that the ring designs of the invention may also be manufactured initially as a closed ring.

1. An external aortic annuloplasty ring for positioning around the circumference of an aortic valve for external aortic root repair or stabilization of the annulus to support the aortic valve, wherein the ring is open-ended and thereby having two opposite open ends, wherein the ring comprises at least two sections with different elastic properties along the perimeter of the ring, and wherein said opposite open ends are suitable for being joined together, so as to form a closed ring around the aortic root.

2-30. (canceled)

31. The annuloplasty ring according to claim 1, wherein the ring comprises a material configured to permit the ring to hold an elastic strain in the range of 1.25-20% strain.

32. The annuloplasty ring according to claim 1, wherein the ring comprises at least three sections having different elastic properties along the perimeter of the ring.

33. The annuloplasty ring according to claim 1, wherein the elastic properties are selected to accommodate the asymmetric dynamics of the aortic annulus of a subject, wherein the non-coronary segment (NC) is less expansible than the right-coronary (RC) and the left-coronary (LC) segments.

34. The annuloplasty ring according to claim 1, wherein the sections having different elastic properties are provided with non-uniform cross-sections.

35. The annuloplasty ring according to claim 1, wherein said annuloplasty ring comprises an elastic core member, and an outer sheath layer, wherein the elastic core member and the outer sheath layer are formed as a band comprising two, opposite open ends, suitable for joining together so as to form a closed ring around the aortic root.

36. The annuloplasty ring according to claim 1, wherein the elastic core member comprises at least two sections having at least two different elastic properties.

37. The annuloplasty ring according to claim 1, wherein at the first open end, the core member has a first length and the sheath has a second length, wherein the second length of the external sheath is longer than the first length of the elastic core member.

38. The annuloplasty ring according to claim 1, wherein at the second open end, the core member has a first length and the sheath has a second length, where the second length of the external sheath is shorter than the first length of the elastic core member.

39. The annuloplasty ring according to claim 1, wherein the open-ended ring is preformed within a predetermined diameter.

40. The annuloplasty ring according to claim 1, wherein the core member is made from silicone or an elastic material having a low stress creep.

41. The annuloplasty ring according to claim 1, wherein the outer sheath layer is made from a medical grade polyester.

42. The annuloplasty ring according to claim 1, wherein the outer sheath layer is made from a woven fabric of medical grade polyester.

43. The annuloplasty ring according to claim 1, wherein the radial thickness of the core member is less that its axial extension.

44. The annuloplasty ring according to claim 43, wherein non-uniform cross-sections are achieved by altering the radial thickness of the elastic core member.

45. The annuloplasty ring according to claim 44, wherein the non-uniform cross-sections comprise more than two sections with different elastic properties along the perimeter of the ring.

46. The annuloplasty ring according to claim 1, wherein the core member is configured to accommodate an aortic valve of a patient, based on a model thereof obtained by a scanning procedure or a measurement of said patient's aortic root.

47. The annuloplasty ring according to claim 46, wherein the core member is made from an additive manufacturing process.

48. A method for manufacturing an external aortic annuloplasty ring for positioning around the circumference of a blood vessel, in particular an open-ended annuloplasty ring for external aortic root repair or stabilization of the annulus, to support the aortic valve, said method comprising:
providing a model or measurement of a blood vessel of a patient,
determining the cross-sectional size of said blood vessel based on said model or measurement, and
manufacturing an elastic core member with a length corresponding to a length of a circumference of said blood vessel, and wherein a cross-sectional diameter of the elastic core member is non-uniform along the length of said elastic core member for providing at least two sections with different elastic properties along the perimeter of the ring.

49. The method for manufacturing an annuloplasty ring according to claim 48, whereby the at least two sections with different elastic properties are formed in the elastic core member in accordance to the model of the blood vessel.

50. The method for manufacturing an annuloplasty ring according to claim 48, whereby the manufacturing of the elastic core member is made by an additive manufacturing process, such as 3D printing.

51. The method for manufacturing an annuloplasty ring according to claim 48, whereby subsequent to manufacturing of the elastic core member, an outer sheath layer is provided around the core member.

52. The method for manufacturing an annuloplasty ring according to claim 48, whereby an external aortic annuloplasty ring for positioning around the circumference of an aortic valve for external aortic root repair or stabilization of the annulus to support the aortic valve is manufactured such that the ring is open-ended and thereby has two opposite open ends, wherein the ring comprises at least two sections with different elastic properties along the perimeter of the ring, and wherein said opposite open ends are suitable for being joined together so as to form a closed ring around the aortic root.

53. A method for surgical repair of an aortic valve of a patient, the aortic valve being exposed to alternating diastolic and systolic phases of a cardiac cycle, the aortic valve having a valve axis and contained within a generally tubular aortic root with an inner surface and an outer surface, the aortic valve including three valve leaflets, the valve leaflets attached to a sigmoid-shaped valve annulus and each having a leaflet free margin, the sigmoid-shaped valve annulus extending circumferentially around the valve axis, the sigmoid-shaped valve annulus extending in height along the valve axis between a nadir portion at a base of the aortic root and a spaced away commissure portion generally at a sinotubular junction of the aortic root, the aortic root also having a subvalvular region located generally below the nadir portion and a supravalvular region located generally above the commissure portion, the aortic root having coronary arteries attached thereto between the subvalvular and supravalvular regions, the aortic root and the valve annulus expanding outwardly away from the valve axis during a cardiac cycle transition from the diastolic phase to the systolic phase and retracting inwardly toward said valve axis during a transition from the systolic phase to the diastolic phase, the valve leaflets movable between a closed configuration in which the leaflet free margins are in an approximated spatial relationship during the diastolic phase and an open configuration in which the leaflet free margins are spaced away from one another during the systolic phase to allow blood flow through the aortic valve generally along a direction parallel to the valve axis, the method comprising:
providing an open ended external aortic annuloplasty ring, wherein the ring is open-ended and thereby having two opposite open ends, wherein the ring comprises at least three sections with different elastic properties along the perimeter of the ring, and
positioning said aortic annuloplasty ring externally on the outer surface and around the aortic root with the two ends of the aortic annuloplasty ring abutting or overlapping each other; and
joining the two ends together to form a subvalvular closed-perimeter flexible structure around the subvalvular region externally of the aortic root, generally adjacent the nadir portion of the valve annulus and below the attachment points of the coronary arteries.

54. The method according to claim 53, whereby the ring is positioned such that the sections of different elastic properties thereof constrain the aortic root with the subvalvular and supravalvular closed-perimeter flexible structures to an anatomically representative geometry that improves coaptation of the leaflet free margins in the diastolic phase of the cardiac cycle.

55. The method according to claim 53, whereby the ring is sutured to the tissue of the subvalvular region externally of the aortic root.

56. A method of treating, inhibiting, or ameliorating an aortic disfunction in a subject comprising: implanting or deploying the annuloplasty ring set forth in claim 1 in a subject thereof.

57. The method of claim 56, further comprising determining the diameter of the aortic root and/or the architecture or topography of the aorta of said subject by measuring the diameter of the aortic root, or using CT scanning or echocardiography and configuring said annuloplasty ring to accommodate said subject's diameter of the aortic root and/or the architecture or topography of the aorta.

58. The method of claim 56 further comprising measuring or evaluating blood flow in said subject after implantation of said annuloplasty ring.

59. The method of claim 56, wherein said annuloplasty ring comprises sections of greater elasticity, which are proximal to regions of the subject's aortic root, which experience greater amounts of flexibility as compared to other regions of said subject's aortic root and, wherein said annuloplasty ring comprises sections of greater rigidity, which are proximal to regions of the subject's aortic root, which experience less or reduced amounts of flexibility as compared to other regions of said subject's aortic root.
